(19) United States
(12) Patent Application Publication
(10) Pub. No.: US 2018/0354900 A1
(43) Pub. Date: Dec. 13, 2018

(54) COMPOSITION CONTAINING ANTICAKING AGENT

(71) Applicant: SUMITOMO SEIKA CHEMICALS CO., LTD., Kako-gun (JP)

(72) Inventors: Katsumi TAKANO, Kako-gun (JP); Takeshi TAKEUCHI, Kako-gun (JP); Hiroyuki SHIRAISHI, Kako-gun (JP)

(73) Assignee: SUMITOMO SEIKA CHEMICALS CO., LTD., Kako-gun (JP)

(21) Appl. No.: 15/781,310

(22) PCT Filed: Dec. 6, 2016
(86) PCT No.: PCT/JP2016/086134

§ 371 Date: Jun. 4, 2018

(30) Foreign Application Priority Data
Dec. 7, 2015 (JP) 2015-238934

(51) Int. Cl. C07C 321/28

(52) U.S. Cl. CPC C07C 321/28; C08G 75/14

(57) ABSTRACT

Provided is a composition that is improved in terms of caking of a disulfide compound. Specifically, the present invention provides a composition containing 0.01 parts by mass or more of an anticaking agent per 100 parts by mass of a disulfide compound represented by formula (1) (wherein R¹ and R² each independently represent a hydrogen atom, a C₁₋₂₀ alkyl group, a hydroxy group, a C₁₋₂₀ alkoxy group, a substituted or unsubstituted amino group, a nitro group, or a halogen atom).

### TECHNICAL FIELD

The present invention relates to a composition comprising a disulfide compound and an anticaking agent for the disulfide compound.

### BACKGROUND ART

Disulfide compounds are widely known to improve adhesion between a metal and a resin and/or enhance the performance of resins by incorporation into various resins. For example, adding a disulfide compound in the process of producing a polyphenylene sulfide (hereinafter abbreviated as PPS) resin has been reported to significantly reduce the sodium content and covalently bonded chlorine content of PPS resin (Patent Literature (PTL) 1).

It has also been reported that adding a disulfide compound to a PPS resin can reduce changes in viscosity and control the viscosity even when melt-kneading is performed under high energy conditions, and can have effects, such as generating a smaller amount of sulfur gas upon heating, and providing a molded product with high mechanical properties and excellent processability (PTL 2).

Further, it has been reported that when an aromatic polyester and a disulfide compound are added to a polyarylene sulfide and kneaded, a polyarylene sulfide resin composition with a high glass transition temperature is obtained (PTL 3).

### CITATION LIST

### Patent Literature

PTL 1: JPS62-241961A
PTL 2: JPH02-286746A
PTL 3: JP2004-182754A
PTL 4: JPH05-246910A
PTL 5: JPH04-045836A
PTL 6: JPS57-203039A

### SUMMARY OF INVENTION

### Technical Problem [0006]

However, disulfide compounds tend to cake easily and reduce work efficiency.

For example, paradichlorobenzene and triethylenediamine are generally known as compounds that easily cake. Such compounds that easily cake usually have sublimability and hygroscopicity and thus have high caking properties. When caking occurs in feeding and/or storage steps, the step of disruption becomes necessary, which significantly reduces work efficiency.

A typical anticaking method for compounds having high caking properties due to sublimability and hygroscopicity is a method comprising adding an additive. For example, a method comprising adding: an organic compound, such as diethyl phthalate, to paradichlorobenzene (PTL 4); a method comprising adding a water-soluble cellulose (PTL 5); and a method comprising adding silica powder to triethylenediamine (PTL 6) have been proposed. The additives disclosed in PTL 4 and PTL 5 are classified into organic compounds, and cannot be used for all purposes. Furthermore, the anticaking method by using an organic compound cannot prevent caking over a long period of time and is thus not effective.

In contrast, disulfide compounds tend to cake easily but do not have high sublimability or hygroscopicity. Disulfide compounds, which have low melting points, are considered to cake easily due to fusion bonding between crystals. High caking properties of disulfide compounds may significantly reduce work efficiency. However, an improvement method therefor has yet to be proposed.

### Solution to Problem

The present inventors conducted extensive research to solve the above problem. As a result, the inventors found that when using a composition containing 0.01 parts by mass or more of an anticaking agent per 100 parts by mass of a disulfide compound, caking can be prevented in supplying, storing, or like steps and work efficiency can be enhanced. The present invention has been accomplished based on this finding.

Specifically, the present invention includes, for example, the following subject matter.

Item 1.

A composition comprising 0.01 parts by mass or more of an anticaking agent per 100 parts by mass of a disulfide compound represented by formula (1):

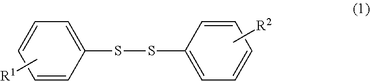

(wherein R¹ and R² each independently (that is, may be the same or different) represent a hydrogen atom, a C₁₋₂₀ alkyl group, a hydroxy group, a Cₗ₋₂₀ alkoxy group, a substituted or unsubstituted amino group, a nitro group, or a halogen atom).

Item 2.

The composition according to Item. 1, wherein the anticaking agent is at least one member selected from the group consisting of silica, thermoplastic resins, and water-soluble inorganic salts.

Item 3.

The composition according to Item 1, wherein the anticaking agent is at least one member selected from the group consisting of thermoplastic resins and water-soluble inorganic salts.

Item 4.

The composition according to Item 1, wherein the anticaking agent is a thermoplastic resin.

Item 5-1.

The composition according to Item 2, wherein the silica has a particle size of 100 nm or less and a specific surface area of 30 m²/g or more.

Item 5-2.

The composition according to Item 2, 3, 4, or 5-1, wherein the thermoplastic resin is at least one member selected from the group consisting of polyphenylene sulfide (PPS), polyether ether ketone (PEEK), polyether ketone (PEK), polycarbonate (PC), and polyether sulfone (PES).

Item 6.

The composition according to Item 1, 2, 3, 4, 5-1, or 5-2, wherein the disulfide compound is represented by formula (1a):

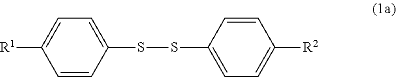

(wherein R¹ and R² are the same or different and represent a hydrogen atom, a methyl group, or an ethyl group).

Item. 7.

The composition according to Item 6, wherein the disulfide compound is diphenyldisulfide.

Item A.

An anticaking agent for a disulfide compound represented by formula (1):

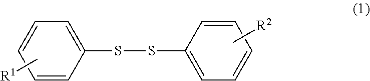

(wherein R¹ and R² independently represent a hydrogen atom, a C₁₋₂₀ alkyl group, a hydroxy group, a C₁₋₂₀ alkoxy group, a substituted or unsubstituted amino group, a nitro group, or a halogen atom).

Item B.

An anticaking method for preventing caking of a disulfide compound, the method comprising mixing at least one member selected from the group consisting of silica, thermoplastic resins, and water-soluble inorganic salts with a disulfide compound represented by formula (1):

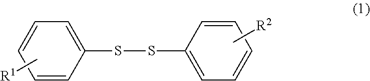

(wherein R¹ and R² independently represent a hydrogen atom, a C₁₋₂₀ alkyl group, a hydroxy group, a C₁₋₂₀ alkoxy group, a substituted or unsubstituted amino group, a nitro group, or a halogen atom). (In the method for preventing caking, at least one member selected from the group consisting of silica, thermoplastic resins, and water-soluble inorganic salts is preferably mixed in an amount of 0.01 parts by mates or more per 100 parts by mass of the disulfide compound.)

### Advantageous Effects of Invention

The present invention can prevent a disulfide compound from caking during supplying, storing, or like steps, and thereby enhance work efficiency.

### DESCRIPTION OF EMBODIMENTS

The disulfide compound of the present invention is represented by formula (1):

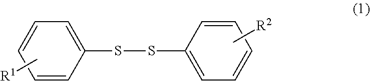

(wherein R¹ and R² independent represent a hydrogen atom, a C₁₋₂₀ alkyl group, a hydroxy atom, a C₁₋₂₀ alkoxy group, a substituted or unsubstituted amino group, a nitro group, or a halogen atom).

The C₁₋₂₀ alkyl group is preferably a C₁₋₁₀ alkyl group, more preferably a C₁₋₆ alkyl group, and even more preferably a C₁₋₄, alkyl group. The alkyl group may be linear or branched and is preferably a linear alkyl group. Specific examples include methyl, ethyl, n-propyl, isopropyl, n-butyl, isobutyl, tert-butyl, and like groups. Among these, methyl, ethyl, and isopropyl are preferable, and methyl and ethyl are more preferable.

The C₁₋₂₀ alkoxy group is preferably a C₁₋₁₀ alkoxy group, more preferably a C₁₋₆ alkoxy group, and even more preferably a C₁₋₄ alkoxy group. The alkoxy group may be linear or branched and is preferably a linear alkoxy group. Specific examples include methoxy, ethoxy, isopropoxy, and like groups. Among these, methoxy is more preferable.

Examples of the substituted or unsubstituted amino group include an amino group, a monomethylamino group, a dimethylamino group, an acetylamino group, and the like. Among these, an amino group (that is, an unsubstituted amino group) and an acetylamino group are preferable.

Examples of the halogen atom include chlorine, fluorine, iodine, and like atoms. Among these, chlorine and fluorine are preferable.

Particularly preferable examples of R¹ and R² include a hydrogen atom, a methyl group, an unsubstituted amino group, an acetylamino group, a nitro group, and a hydroxy group.

R¹ and R² are independent as described above. In other words, R¹ and R² may be the same or different. R¹ and R² are preferably the same.

R¹ and R² are preferably substituents at position 4 and position 4′, respectively. Specifically, the disulfide compound according to the present invention is preferably represented by formula (1a)

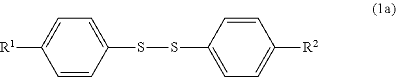

(wherein R¹ and R² are as defined above).

Among the disulfide compounds of the present invention, particularly preferable compounds are, for example, compounds represented by formula (1a) wherein R¹ and R² are the same substituent and each represent a hydrogen atom, a methyl group, an unsubstituted amino group, an acetylamino group, a nitro group, or a hydroxy group. Specific examples include diphenyldisulfide, 4,4′-diaminodiphenylsulfide, 4,4′-dimethyldiphenyldisulfide, 4,4′-diacetylaminodiphenyldisulfide, 4,4′-dinitrodiphenyldisulfide, 4,4′-dihydroxydiphenyl disulfide, and the like. Diphenyldisulfide is more preferable.

Examples of the anticaking agent include silica, thermoplastic resins, water-soluble inorganic salts, and the like. In view of preventing caking without becoming an impurity in the produced resin, the anticaking agent is preferably a thermoplastic resin or a water-soluble inorganic salt, and is more preferably a thermoplastic resin. Such anti-caking agents can he used singly or in a combination of two or more.

The silica preferably has a small particle size. For example, the silica preferably has a particle size of 100 nm or less, and more preferably 50 nm or less. The lower limit of the particle size is not particularly limited, and may be, for example, 5 nm or more. The particle size herein refers to a primary particle size. The average primary particle size is obtained by determining the particle size distribution of silica particles by transmission electron microscope observation and calculating the average particle size. In particular, when a commercially available product, such as Aerosil series (Evonik Degussa GMBH) or Reorosil (produced by Tokuyama Corporation), is used as silica, the particle size shown in the catalog is the particle size referred to herein.

The silica preferably has a large specific surface area. For example, the silica preferably has a specific surface area of 30 m²/g or more, more preferably 40 m²/g or more, and still more preferably 50 m²/g or more. The upper limit of the specific surface area is not particularly limited, and may be, for example, 500 m²/g or less. The specific surface area herein refers to the specific surface area determined by the BET adsorption method (according to JIS Z8830).

Examples of silica include combustion silica, which is a dry-process silica; precipitated silica, which is a wet-process silica; gel processed silica; and the like. Specific examples of combustion silica include Aerosil (produced by Evonic), CAB-O-SIL (produced by Cabot Corporation), HDK (produced by Asahi Kasei Corporation), Reolosil (produced by Tokuyama Corporation), and the like. Examples of precipitated silica include Nipsil (produced by Nihon Silica Kogyo, Co., Ltd.), Ultrasil (produced by Evonic), Tokusil (produced by Tokuyama Corporation), and the like. Examples of gel processed silica include Sylysia (produced by Fuji Silysia Chemical Ltd.), Syloid (produced by W.R. Grace & Co.), Nipgel (produced by Nippon Silica Kogyo K.K.), and the like. Among these, combustion silica has a very small particle size of 5 to 50 nm and a very large specific surface area of 50 to 400 m²/g, and has good fluidity. Therefore, addition of a small amount of combustion silica is expected to prevent caking of a disulfide compound by coating the surface of the disulfide compound with the combustion silica. Accordingly, Aerosil (registered trademark) and Reorosil (registered trademark), which are combustion silica, etc., are preferable. Such silica can be used singly or in a combination of two or more.

When the anticaking agent is silica, the anticaking agent content is 0.01 parts by mass or more, and preferably 0.01 to 5 parts by mass, per 100 parts by mass of the disulfide compound. The lower limit of the anticaking agent content is preferably 0.05 parts by mass. The upper limit of the anticaking agent is preferably' 5 parts by mass, more preferably 3 parts by mass, and still more preferably 1 part by mass. The anticaking agent content is 0.05 to 5 parts by mass, more preferably 0.05 to 3 parts by mass, and still more preferably 0.05 to 1 part by mass.

Examples of the thermoplastic resin include polyphenylene sulfide (PPS), polyether ether ketone (PEER), polyether ketone (PEK), polycarbonate (PC), polyether sulfone (PES), polyvinyl chloride (PVC), polystyrene (PS), polypropylene (PP), ABS resin (ABS), polyamide (PA), phenolic resin (PF), melamine resin KM epoxy resin (EP) polysulfone (PSU), and the like. Among these, polyphenylene sulfide (PPS), polyether ether ketone (PEER), polyether ketone (PEK), polycarbonate (PC), and polyethersulfone (PES) are preferable. A terminally halogenated thermoplastic resin is preferably used because the disulfide compound can be used as a resin additive for removing halogen group(s) from the terminally halogenated thermoplastic resin. Examples of such halogen groups include fluoro, chloro, bromo, iodine, and like groups fluoro, chloro, and bromo groups are preferable, and fluoro and chloro groups are more preferable. A composition comprising the disulfide compound that contains a terminally halogenated thermoplastic resin as an anticaking agent is preferable because it can be used as is in the halogen removal step.

For example, the Phillips Petroleum method is a method of synthesizing a polyphenylene sulfide by polycondensation of p-dichlorobenzene and sodium sulfide in an amide polar catalyst solvent under high-temperature and high-pressure conditions of about 200 to 290° C. The Phillips Petroleum method is preferable because the polyphenylene sulfide obtained by this method theoretically contains chloro group (s) at its end(s), and the disulfide compound can be used to remove the chloro group(s).

Poly (paraphenylene sulfide) [poly(1,4-phenylene sulfide)] is particularly preferable as polyphenylene sulfide (PPS). The thermoplastic resins can be used singly or in a combination of two or more.

When a thermoplastic resin as described above is used as the anticaking agent for a disulfide compound and, for example, when a disulfide compound containing the anticaking agent is used as a resin additive for enhancing the performance of a PPS resin, the PPS resin is preferably used as the anticaking agent.

When a thermoplastic resin is used as an anticaking agent to be added to a disulfide compound, a resin whose performance is intended to be improved by adding the disulfide compound is preferably used as the anticaking agent. When such a resin is mixed with the disulfide compound, the disulfide compound can be prevented from caking and the disulfide compound, which is prevented from caking, can be added during the production of the resin, thereby enhancing: the resin performance. In this case, since the resin used as an anticaking agent and the resin whose performance is intended to be improved are the same, a resin composition in which the anticaking agent does not become an impurity can be provided. (In other words, when “a composition comprising an anticaking agent and a disulfide compound” is mixed with a resin whose performance is intended to be improved, the “anticaking agent” and “the resin whose performance is intended to be improved” are preferably the same).

When a thermoplastic resin is used as the anticaking agent, the anticaking agent content is 0.01 parts by mass or more, preferably 0.1 parts by mass or more, more preferably 0.5 parts by mass or more, even more preferably1 to 100 parts by mass, still even more preferably 3 to 50 parts by mass, and particularly preferably 3 to 30 parts by mass, per 100 parts by mass of the disulfide compound.

Examples of the water-soluble inorganic salt include sodium chloride (NaCl), potassium chloride (KCl), magnesium chloride (MgCl₂), sodium bromide (NaBr), sodium sulfate (Na₂SO₄), potassium sulfate (K₂SO₄), magnesium sulfate (MgSO₄), ammonium chloride (NH₄Cl), sodium carbonate (Na₂CO₃) potassium carbonate (K₂CO₃), sodium hydrogen carbonate (NaHCO₃), potassium hydrogen carbonate (KHCO₃), and the like. Among these, NaCl, KCl, and Na₂SO₄ are preferable because they can be easily removed from the system (i.e., separated from the disulfide compound) after addition. Water-soluble inorganic salts can be used singly or in a combination of two or more.

When a water-soluble inorganic salt is used as the anticaking agent, the anti-caking agent content is 0.01 parts by mass or more, preferably 0.1 parts by mass or more, more preferably 0.5 parts by mass or more, even more preferably 1.0 part by mass or more, and still even more preferably 2.0 parts by mass or more, per 100 parts by mass of the disulfide compound. The upper limit of the anticaking agent content is not particularly limited. For example, the upper limit is preferably 50 parts by mass or less (that is, preferably 0.01 to 50 parts by mass), and more preferably 10 parts by mass or less.

As described above, the disulfide compound may be used as a resin additive for improving the performance of various resins. When silica is used as an anticaking agent for the disulfide compound, silica may become an impurity in the resin to which the disulfide compound is added. Therefore, the anticaking agent used in the present invention is preferably a thermoplastic resin or a water-soluble inorganic salt, and is more preferably a thermoplastic resin.

The method for producing the composition of the present invention is not particularly limited. Examples include a method comprising stirring a disulfide compound and an anticaking agent while drying using an evaporator; a method comprising stirring by using a dryer, such as a conical dryer, a Nauta dryer, or a vibrating flow dryer; a method comprising mixing using a powder mixer, such as a tumbler mixer or a drum mixer.

The present invention includes an anticaking agent for a specific disulfide compound, for example, as described above in Item A. The present invention further includes a method for preventing caking of a specific disulfide compound, for example, as described above in Item B. The disulfide compound, silica, thermoplastic resin, and water-soluble inorganic salt used in the anticaking agent or the anticaking method, and their blending ratio may be the game as described above.

### EXAMPLES

### Example 1

Twenty grams of diphenyl disulfide and. 0.2 g of Aerosil 200 (primary particle size: 12 nm, specific surface area: 200±25 m²/g) were weighed into a recovery (pear-shaped) flask and rotated and stirred using an evaporator for 1 hour. The obtained sample was transferred to a 50-mL sample bottle and stored at 25° C. After 1 week, the sample bottle was turned on its side. It was confirmed that the diphenyl disulfide flowed and no caking occurred.

### Example 2

Twenty grams of diphenyl disulfide and 0.02 g of Aerosil 200 were weighed into a recovery (pear-shaped) flask and rotated and stirred using an evaporator for 1 hour. The obtained sample was placed into a 50-mL sample bottle and stored at 25° C. After 1 week, the sample bottle was turned on its side. It was confirmed that the diphenyl disulfide flowed and no caking: occurred.

### Example 3

Twenty grams of diphenyl disulfide and 0.01 g of Aerosil 200 were weighed into a recovery (pear-shaped) flask and rotated and stirred using an evaporator for 1 hour. The obtained sample was placed into a 50-mL sample bottle and stored at 25° C. After 1 week, the sample bottle was turned on its side. It was confirmed that diphenyl disulfide flowed and but partially caked in lumps. The force at which the lumps collapsed was determined using a compression tester. The lumps were confirmed to collapse at 0.26 kg/cm².

### Example 4

The procedure was carried out in the same manner as in Example 1 except that 0.06 g of Aerosil R972 (primary particle size: 16 nm, specific surface area: 110±20 m²/g) was added as an anticaking agent. The obtained sample was stored at 25° C. After 1 week, the sample bottle was turned on its side. It was confirmed that the diphenyl disulfide flowed and no caking occurred.

### Example 5

The procedure was carried out in the same manner as in Example 1 except that 0.06 g of Aerosil RX50 (primary particle size: 30 nm, specific surface area: 35±10 m/g) was added as an anticaking agent. The obtained sample was stored at 25° C. After 1 week, the sample bottle was turned on its side. It was confirmed that the diphenyl disulfide flowed and no caking occurred.

### Example 6

The procedure was carried out in the same manner as in Example 1 except that 0.06 g of Aerosil RY300 (primary particle size: 7 nm, specific surface area: 125±15 m²/g) was added as an anticaking agent. The obtained sample was stored at 25° C. After 1 week, the sample bottle was turned on its side. It was confirmed that the diphenyl disulfide flowed and no caking occurred.

### Example 7

Twenty grams of diphenyl disulfide and 4.0 g o a PPS resin (poly(1,4-phenylene sulfide) Aldrich's catalog No. 182354, primary particle size: 11000 nm) were weighed into a recovery (pear-shaped) flask and rotated and stirred using an evaporator for 1 hour. The obtained sample was transferred to a 50-mL sample bottle and stored at 25° C. After 1 week, the sample bottle was turned on its side. It was confirmed that the diphenyl disulfide flowed and no caking occurred.

### Example 8

Twenty grams of diphenyl disulfide and 2.0 g of a PPS resin (the same resin as in Example 7) were weighed into a recovery (pear-shaped) flask and rotated and stirred using an evaporator for 1 hour. The obtained sample was transferred to a 50-mL sample bottle and stored at 25° C. After 1 week, the sample bottle was turned on its side. It was confirmed that the diphenyl disulfide flowed and no caking occurred.

### Example 9

Twenty grams of diphenyl disulfide and 0.2 g of a PPS resin (the same resin as in Example 7) were weighed into a pear-shape flask and rotated and stirred using an evaporator for 1 hour. The obtained sample was transferred to a 50-mL sample bottle and stored at 25° C. After 1 week, the sample bottle was turned on its side. It was confirmed that the diphenyl disulfide flowed but partially caked in lumps. The consolidation strength of the caked lumps was determined using a compression tester and confirmed to be 0.1 kg/cm².

### Comparative Example 1

### Caking of DPDS Alone

Twenty grams of diphenyl disulfide (DPDS) was weighed into a 50-mL sample bottle and stored at 25° C. After 4 hours, the diphenyl disulfide had caked. Even when the sample bottle was turned on its side or placed upside down, the diphenyl disulfide maintained its initial state and had caked. The consolidation strength of the caked lumps was determined using a compression tester and confirmed to be 0.5 kg/cm.².

TABLE 1

| | | Content | | | Caking | |
|---|---|---|---|---|---|---|
| | Anticaking | (parts by | Storage | | strength | |
| | agent | mass) | time | Fluidity | (kg/cm²) | Evaluation |
| Example 3 | Aerosil 200 | 0.05 | 1 week | Partial | 0.26 | B |
| | | | | caking | | |
| Example 2 | Aerosil 200 | 0.1 | 1 week | No caking | — | A |
| Example 1 | Aerosil 200 | 1 | 1 week | No caking | — | A |
| Example 4 | Aerosil R972 | 0.3 | 1 week | No caking | — | A |
| Example 5 | Aerosil RX50 | 0.3 | 1 week | No caking | — | A |
| Example 6 | Aerosil RY-300 | 0.3 | 1 week | No caking | — | A |
| Example 7 | PPS | 20 | 1 week | No caking | — | A |
| Example 8 | PPS | 10 | 1 week | No caking | — | A |
| Example 9 | PPS | 1 | 1 week | Partial | 0.1 | B |
| | | | | caking | | |
| Comparative | None | — | 4 hours | No | 0.5 | C |
| Example 1 | | | | fluidity | | |

Evaluation Criteria
A: No caking occurred (fluidity existed).
B: Partial caking was observed. The lumps collapsed when a force of less than 0.5 kg/cm² was applied
C: Entire caking was observed, or a force of 0.5 kg/cm² or more was necessary to collapse the lumps formed.

1. A composition comprising 0.01 parts by mass or more of an anticaking agent per 100 parts by mass of a disulfide compound represented by formula (1): 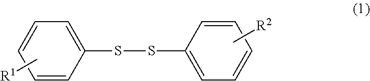
wherein R¹ and R² each independently represent a hydrogen atom, a C₁₋₂₀ alkyl group, a hydroxy group, a C₁₋₂₀ alkoxy group, a substituted or unsubstituted amino group, a nitro group, or a halogen atom.

2. The composition according to claim 1, wherein the anticaking agent is at least one member selected from the group consisting of silica, thermoplastic resins, and water-soluble inorganic salts.

3. The composition according to claim 1, wherein the anticaking agent is at least one member selected from the group consisting of thermoplastic resins and water-soluble inorganic salts.

4. The composition according to claim 1, wherein the anticaking agent is a thermoplastic resin.

5. The composition according to claim 2, wherein the thermoplastic resin is at least one member selected from the group consisting of polyphenylene sulfide (PPS), polyether ether ketone (PEEK), polyether ketone (PEK), polycarbonate (PC), and polyether sulphone (PES).

6. The composition according to claim 1, wherein the disulfide compound is a compound represented by formula (1a): 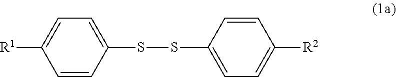
wherein R¹ and R² are the same or different and each represents a hydrogen atom, a methyl group, or an ethyl group.

7. The composition according to claim 6, wherein the disulfide compound is diphenyl disulfide.

8. An anticaking method for preventing caking of a disulfide compound, comprising mixing the disulfide compound with at least one member selected from the group consisting of silica, thermoplastic resins, and water-soluble inorganic salts, the disulfide compound being represented by formula (1): 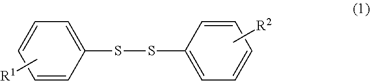
wherein R¹ and R² independently represent a hydrogen atom, a C₁₋₂₀ alkyl group, a hydroxy group, a C₁₋₂₀ alkoxy group, a substituted or unsubstituted amino group, a nitro group, or a halogen atom.
